(19) United States
(12) Patent Application Publication
(10) Pub. No.: US 2023/0277365 A1
(43) Pub. Date: Sep. 7, 2023

(54) Molar Shim System for Posterior Bite Registration

(71) Applicant: Allen J. Moses, Indian Wells, CA (US)

(72) Inventor: Allen J. Moses, Indian Wells, CA (US)

(21) Appl. No.: 17/687,092

(22) Filed: Mar. 4, 2022

(51) Int. Cl. A61F 5/56

(52) U.S. Cl. CPC A61F 5/566

(57) ABSTRACT

The present invention is a posterior molar shim system that allows an oral sleep appliance clinician to incorporate the parameters of vertical, lateral, protrusive and slant into the process of designing an effective oral sleep appliance. The molar shim system is comprised of square-shaped, rigid shims having between 2.0 mm-8.0 mm thicknesses and having two protrusions (herein referred to as ‘alignment pegs’) on one side and two matching apertures on another side. Said pegs and apertures allow the shims to be stacked in a stable, accurate, vertical, friction-fit manner. One side of the shims also has a slot to receive a rectangular arch-wire that connects a set of shims for positioning inside a mouth. An object of the system is to provide a means of posterior inter-arch stabilization during bite registration to reduce slant angle as part of an oral sleep appliance design that minimizes sleep apnea more effectively.

### FIELD OF THE INVENTION

The present invention generally relates to oral sleep appliances. More specifically, it relates to a posterior adjustable shim system for oral sleep appliance design.

### BACKGROUND

Dental sleep medicine involves the use of oral appliances for the treatment of sleep-related breathing disorders such as obstructive sleep apnea and snoring. Obstructive sleep apnea (OSA) is a common sleep disorder characterized by multiple apnea events, due to short-duration blockages of the upper airway during sleep, resulting in decreased airflow to the lungs. OSA in general decreases the quality of life of the affected adults. In fact, it is a life-threatening condition and has been reported as a risk factor for depression, hypertension, cardiovascular diseases, reduced vitality, impotence, diabetes, obesity, and occupational and motor vehicle accidents. Classic symptoms of OSA include loud snoring and arousal/awakening from sleep, daytime sleepiness, tiredness, and lack of concentration. The scientific literature reports that mandibular advancement appliances have been successfully used as an alternative option for treating mild-to-moderate OSA in adults who do not tolerate the continuous positive airway pressure (CPAP) therapy. Most intraoral appliances hold the jaw forward which dilates and stents open the airway. The field of dental sleep medicine continues to evolve. U.S. Pat. No. 7,364,429B22004 granted to Juan disclosed methods and apparatus for determining horizontal and vertical plane orientation for construction of dental prostheses. United States Patent No. US20070209666A1 granted to Halstrom teaches of mandible positioning devices. United States Patent No. US20070264609A1 granted to Zebris Medical Gmbh teaches of a method and apparatus for the 3-Dimensional analysis of movement of the tooth surfaces of the maxilla in relation to the mandible. U.S. Pat. No. 5,846,212A granted to the Therabite Corporation disclosed a passive mandible translator.

U.S. Pat. No. 5,678,567A granted to Thornton disclosed an apparatus for adjusting a dental device. U.S. Pat. No. 5,794,627A granted to Frantz teaches of a disposable Mandibular advancement appliance. U.S. Pat. No. 5,816,799A granted to Parker disclosed an intraoral orthopedic appliance adjustment apparatus. U.S. Pat. No. 5,409,017A granted to the University of British Columbia Mandible repositioning appliance. U.S. Pat. No. 5,154,609A granted to George disclosed an instrument for registration of the dental bite. U.S. Pat. No. 4,602,905A granted to Keefe teaches of a dental impression registration device. U.S. Pat. No. 4,472,140A granted to Lustig disclosed a bite registration device. U.S. Pat. No. 8,226,407B2 granted to Hanewinkel disclosed a Mandibular Manipulator. U.S. Pat. No. 8,646,454 B1 granted to Moses disclosed an oral appliance and methods of making oral appliances involving anterior stabilization shims. United States Patent No. US 20220015878A1 granted to Cowburn disclosed a method and system for acquiring data from an individual for preparing a 3d model. U.S. Pat. No. 9,615,963B2 granted to Horchover is an anterior jaw stabilization system for fitting an oral sleep appliance to maintain maximum airway dimension. While all of these inventions are noteworthy, they appear to fall short of maximizing airway potential. In fact, increasing vertical dimensions in the anterior area of the mouth does not allow for any proportional increase in the posterior area of the mouth where the airway dilation and clearance is most desired. If the goal of typical anterior stabilization methods is to achieve the necessary posterior clearance, it may be necessary to introduce so much vertical in the anterior of the sleep appliance that the lips cannot comfortably stay closed, and mouth breathing becomes a sequelae—which is undesirable. What is needed is a posterior adjustable shim system that allows for vertical, lateral, protrusive distances as well as eliminating or greatly reducing slant when determining an inter-arch jaw relationship.

The present invention, a posterior shim system, positioned in the molar areas stabilizes or offers resistance against posterior over-closure and eliminates or greatly reduces slant. The stenting effect is gained in the posterior of the mouth where it is necessary for airway dilation. The resulting maxillary and mandibular occlusal planes are very close to parallel. The consequence of using anterior stabilization for an inter-arch jaw registration is that increasing vertical dimension in the anterior does not gain proportional increase in the posterior of the mouth, where the airway dilation and clearance is most important. Mandibular musculature is coordinated and balanced by a central pattern generator and curbed by proprioceptive reflexes. This neural feedback mechanism is subject to anomalies that lead to an imbalance of these delicate, normally harmonized muscles. Displacement of the mandible by an anterior stabilization shim to register the inter-arch jaw position for an intraoral sleep apnea appliance is one example. The position of an anterior stabilization device disrupts the normal harmonious firing pattern of the facial muscle complex, their proprioceptors, tendons and the temporomandibular joint. The neural message from the powerful elevator muscles situated in the molar area is lack of resistance to contraction, so they continue to contract, creating an abnormal inter-arch slant toward the posterior. Slant is not a good thing. Also, when anterior stabilization is used to achieve the necessary posterior airway clearance, it may be necessary to introduce so much vertical in the anterior of the sleep appliance that the lips cannot comfortably stay closed, and mouth breathing becomes a sequelae.

### SUMMARY OF THE INVENTION

The device herein disclosed and described provides a solution to the shortcomings in the prior art through the disclosure of a posterior molar shim system. An object of the system is to allow dental health providers to design more effective oral sleep appliances for their patients. The system incorporates a means to eliminate or minimize slant in the inter-arch jaw position over conventional anterior shim systems during bite registration. The current disclosure focuses on widening the bite in the posterior of the jaw with shims during bite registration instead. Thereby minimizing the slant angle of the jaw so that oral sleep appliances have the ability to further dilate the airway and thereby minimizing sleep apnea more effectively.

Another object of the invention is to provide a means to easily increase the thickness of shims. Shims come in different sizes and are selectively interconnected or stacked by means of small pegs and apertures.

Another object of the invention is to allow users to maintain multiple shim alignment during bite registration. The aforementioned shim pegs friction fit together and maintain the selected positions together during this process preventing any lateral movement.

Another object of the invention is to allow shims to be easily installed into and removed from a patient's mouth. The shims are connected by a shim coupler made of a thin, rectangular-shaped wire or is molded as a single piece into the shims.

Another object of the invention is to allow shim sizes to be easily identified. Each shim is embossed with thickness indicia that is easily visible.

Another object of the invention is to maximize comfort to the patient during use. The corners of the shims are filleted and the shim coupler is very thin for maximum comfort for the patient.

It is briefly noted that upon a reading this disclosure, those skilled in the art will recognize various means for carrying out these intended features of the invention. As such it is to be understood that other methods, applications and systems adapted to the task may be configured to carry out these features and are therefore considered to be within the scope and intent of the present invention, and are anticipated. With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. The objects, features, and advantages of the present invention, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF THE FIGURES

The accompanying drawings, which are incorporated herein and form a part of the specification, illustrate some, but not the only or exclusive, examples of embodiments and/or features.

FIG. 1 shows a top perspective view of the device.

FIG. 2 shows close up, top perspective view of different modular shim sizes.

FIG. 3 shows close up, top perspective view of modular shims.

FIG. 4 shows close up, top perspective view of modular shims stacked.

FIG. 5 shows various plan and elevation views of the modular shims.

FIG. 6 shows slant angles in relation to airway dilation.

FIG. 7 shows a perspective view of another embodiment of the modular shims.

FIG. 8 shows a representative view of the posterior modular shim method.

FIG. 9 shows a representative view of the elevator muscles.

Other aspects of the present invention shall be more readily understood when considered in conjunction with the accompanying drawings, and the following detailed description, neither of which should be considered limiting.

### DETAILED DESCRIPTION OF FIGURES

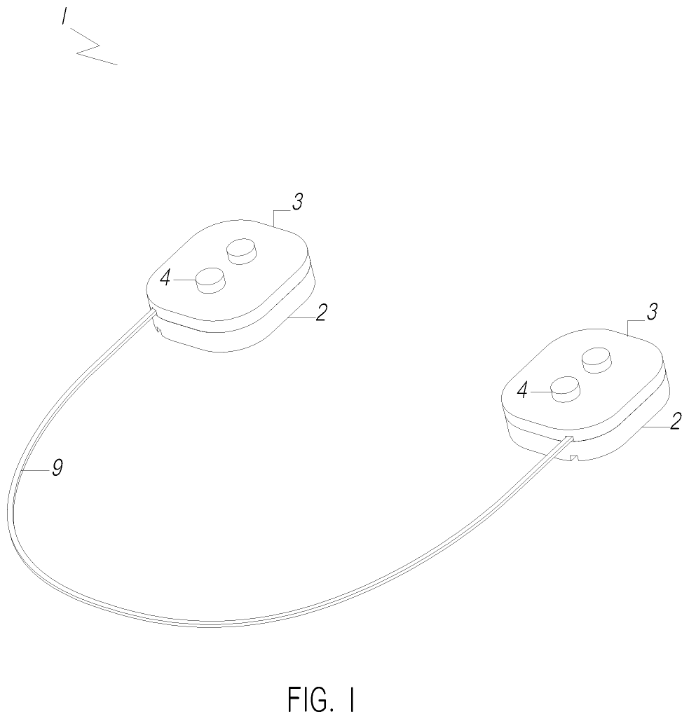

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation. Conventional components of the invention are elements that are well-known in the prior art and will not be discussed in detail for this disclosure. FIG. 1 shows a top perspective view of a preferred embodiment of posterior molar shim system 1 comprised of a plurality of rectangular-shaped, thick and thin shims (2 and 3 respectively) connected by means of shim coupler 9. Said thick and thin shims 2 and 3 being made of a rigid material including but not limited to plastic, epoxy and the like and having curved corners 5 for patient comfort. Shim coupler 9 is comprised of a rigid, arc-shaped member made of a metal and having a square profile to minimize twisting of thick and thin shims 2 and 3. Said profile having dimensions that include but are not limited to 0.46 mm wide and 0.56 mm high. Other embodiments of shim coupler 9 can have other shapes and different profiles and can even be molded into the same material as thick and thin shims 2 and 3. Said thick shims 2 having a thickness of at least but not limited to 2.0 mm and said thin shims 3 having a thickness of at least but not limited to 1.5 mm. Other embodiments can include the aforementioned thick and thin shims (2 and 3 respectively) having different shapes including but not limited to squares, circles and the like. The molar shim system having essentially smooth undersurfaces in which the mandible can slide to duplicate and register protrusive and lateral jaw movement whose placement between the upper and lower molar teeth will eliminate or greatly reduce the possibility of slant. This system is capable of accurately registering the inter-arch jaw position resulting from asymmetric jaw movement.

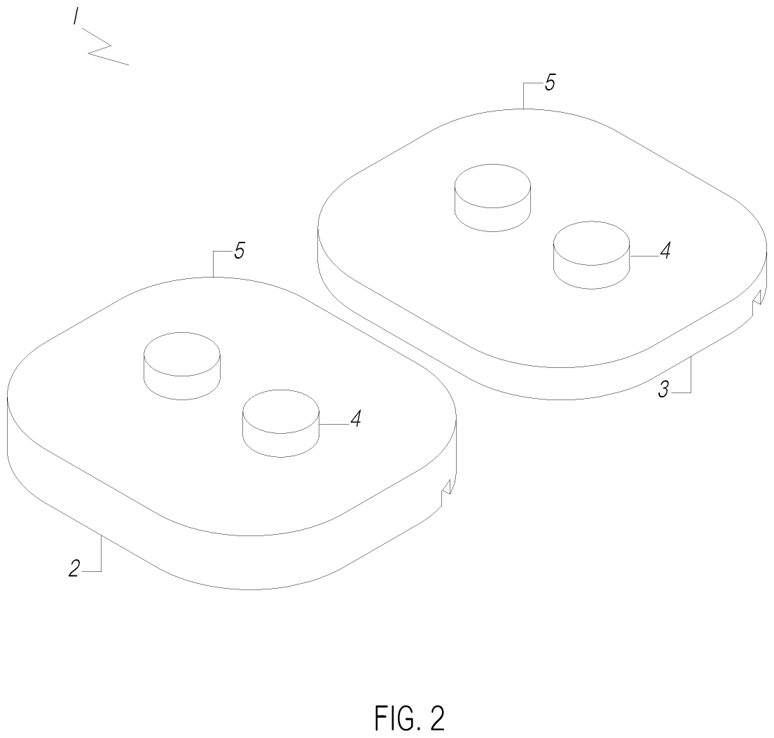

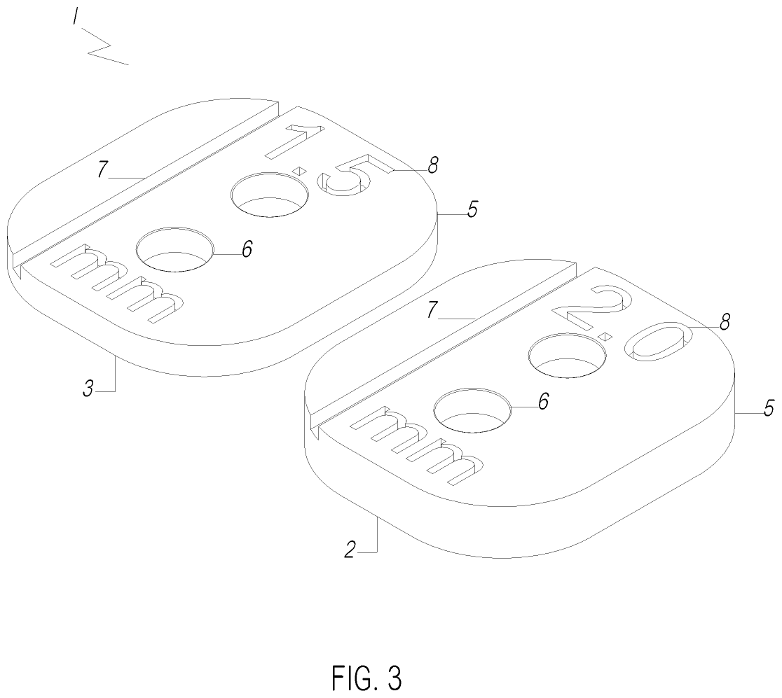

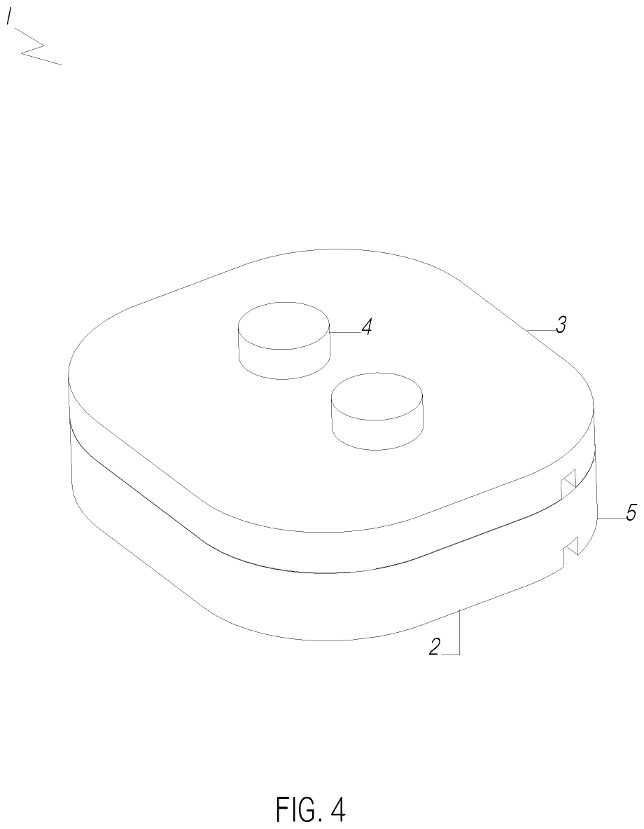

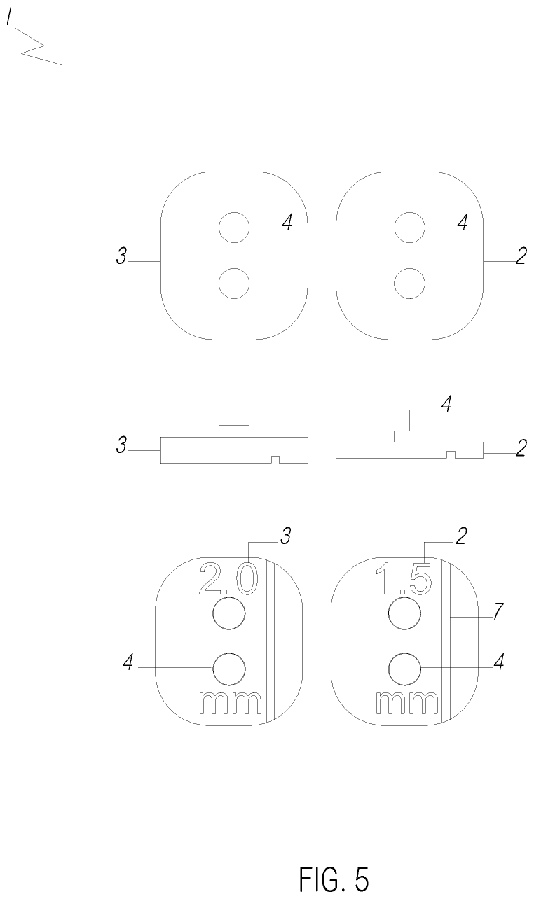

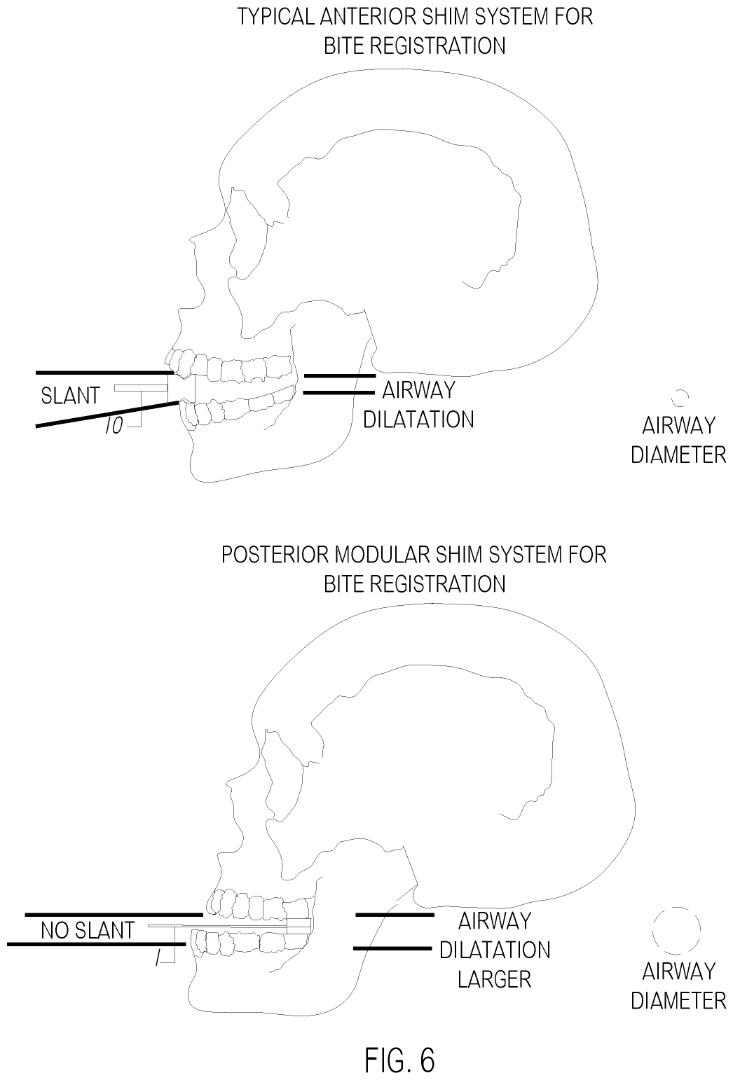

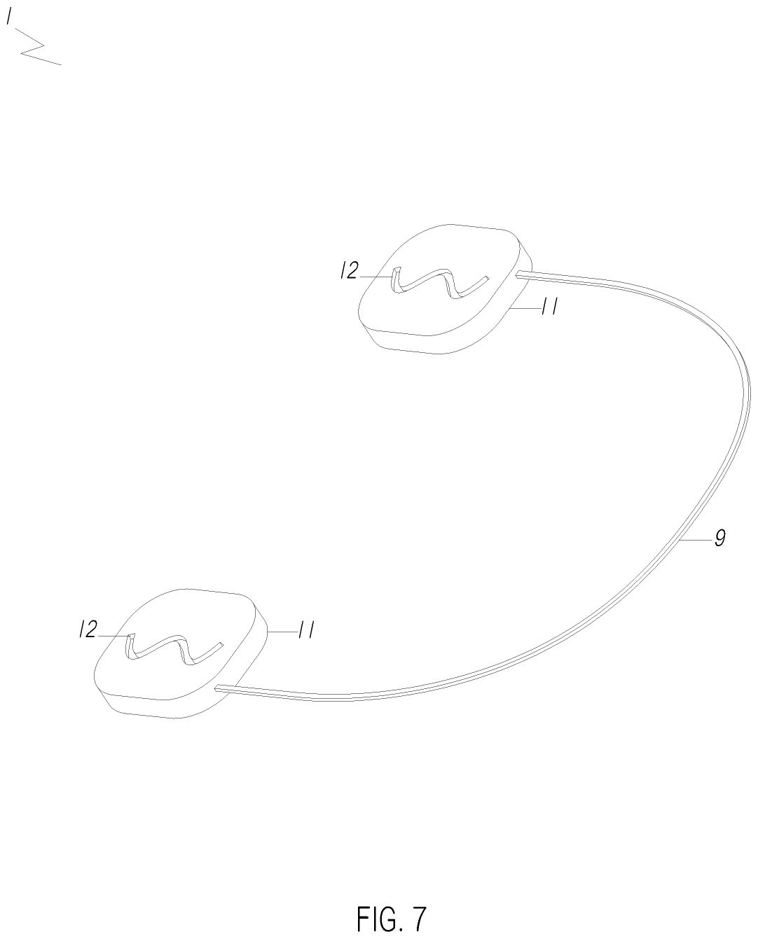

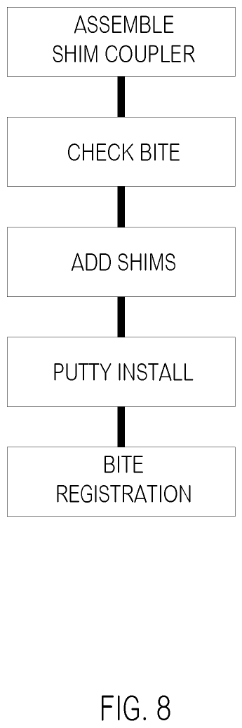

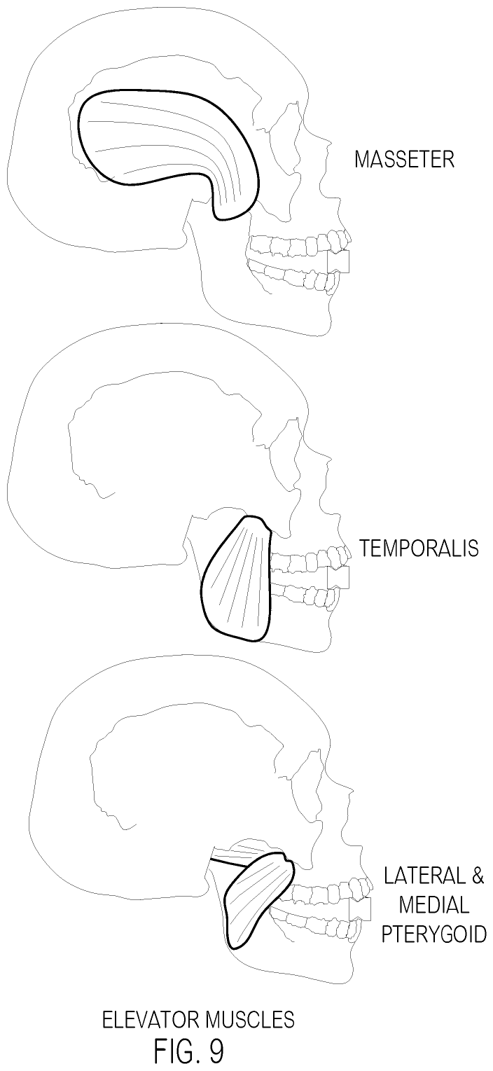

FIG. 2 shows a close up, top perspective view of different modular shim sizes along with at least two cylindrical-shaped, alignment pegs 4 configured along the proximal, longitudinal axis of thick and thin shims (2 and 3 respectively). Said alignment pegs 4 having a height that includes but is not limited to 1.0 mm. Other embodiments can include alignment pegs 4 having different shapes including but not limited to rectangular, triangles and the like. FIG. 3 shows close up, top perspective view of modular shims having at least two alignment apertures 6 configured along the proximal, longitudinal axis of thick and thin shims (2 and 3 respectively) and having a diameter slightly larger than the alignment pegs 4 to allow for a friction fit there between. The alignment apertures 6 engage the alignment pegs 4 for shim stacking. The figure also showing shim thickness indicia 8 embossed onto shim surfaces as well as at least two shim coupler slots 7 configured distally along the longitudinal axis of thick and thin shims (2 and 3 respectively) and configured to have dimensions slightly larger than shim coupler 9 thereby also allowing for a friction fit there between. FIG. 4 shows close up, top perspective view of modular shims stacked wherein the alignment pegs 4 being received by alignment apertures 6 allowing for a friction fit there between. FIG. 5 shows various plan and elevation views of the modular shims 2 and 3. FIG. 6 shows a representative view slant angles in relation to airway dilation. During conventional, bite registrations with shim systems the focus is mainly on separating the jaw at the anterior portion of the mouth to achieve airway dilation. The current disclosure focuses on the posterior of the mouth when separating the jaw during bite registration. This posterior focus leads to further, desirable, dilation of the airway as shown in FIG. 6. FIG. 7 shows a perspective view of another embodiment of the modular shims without pegs 4 and being configured with a curving slot 12 on one side (for tooth grip) and having thicknesses between but not limited to 2 mm to 5 mm. FIG. 8 shows a representative view of the posterior modular shim method that includes but is not limited to the following steps: assembling terminal ends of shim coupler 9 into shim coupler slot 7 of thick and thin shims (2 and 3 respectively); inserting the posterior molar shim system 1 into a patient's mouth until shims are positioned over the rear molars; checking the bite slant and adding additional shims if necessary (to minimize said slant); installing impression putty onto thick and thin shims 1 and 2; and having a patient bite down to complete the bite registration process. FIG. 9 illustrates how attachment of the elevator muscles in the posterior of the mouth can result in slant by use of an anterior stabilization device.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner. While all of the fundamental characteristics and features of the invention have been shown and described herein, with reference to particular embodiments thereof, a latitude of modification, various changes and substitutions are intended in the foregoing disclosure.

1. A molar shim system for oral sleep appliance design comprised of the following parts:
a) shims for minimizing the slant angle of the jaw so that oral sleep appliances have the ability to dilate an airway thereby minimizing sleep apnea; and
b) a shim coupler.

2. A molar shim system with smooth undersurfaces in which a mandible can slide to duplicate and register protrusive and lateral jaw movement.

3. A molar shim system whose placement between upper and lower molar teeth will eliminate or greatly reduce the possibility of slant.

4. A molar shim system capable of accurately registering an inter-arch jaw position resulting from asymmetric jaw movement.

5. The molar shim system for oral sleep appliance design of claim 1, wherein the shims being made of a rigid material including but not limited to plastic, epoxy and having curved corners for patient comfort.

6. The molar shim system for oral sleep appliance design of claim 1, wherein the molar shims have at least two cylindrical-shaped, alignment pegs configured along a proximal, longitudinal axis of said shims.

7. The molar shims of claim 6, wherein the molar shims having at least one alignment apertures configured along the proximal, longitudinal axis of said molar shims that engage said alignment pegs on molar shims for stacking.

8. The molar shims of claim 7, wherein the alignment apertures having a diameter slightly larger than the alignment pegs to allow for a friction fit there between.

9. The molar shim system for oral sleep appliance design of claim 1, wherein the shims having shim thickness indicia embossed onto shim surfaces.

10. The molar shim system for oral sleep appliance design of claim 1, wherein the shims having at least one shim coupler slot configured distally along a longitudinal axis of the shims.

11. A method of minimizing slant during bite registration for oral sleep appliance design comprising the following steps:
a) providing the molar shim system of claim 1;
b) assembling terminal ends of the shim coupler into shim coupler slots of the shims;
c) inserting the molar shim system into a patient's mouth until the shims are positioned over rear molars;
d) checking a bite slant and adding additional shims if necessary;
e) installing impression putty onto thick and thin shims; and
f) having the patient bite down to complete the bite registration process.
